(19) United States
(12) Patent Application Publication
(10) Pub. No.: US 2009/0269375 A1
(43) Pub. Date: Oct. 29, 2009

(54) Tanning compositions containing juice concentrate

(76) Inventor: Diahne Patnode, Chandler, AZ (US)

Correspondence Address: KRAMER & AMADO, P.C., 1725 DUKE STREET, SUITE 240, ALEXANDRIA, VA, 22314, US

(21) Appl. No.: 12/149,054

(22) Filed: Apr. 25, 2008

(51) Int. Cl. A61K 8/97; A61K 8/06; A61Q 17/04

(52) U.S. Cl. 424/401; 424/59

(57) ABSTRACT

A topical tanning composition comprises an aqueous continuous phase comprising water; and at least one fruit or vegetable juice; at least one optional compound selected from the group consisting of tanning accelerators, bronzers, and a mixture thereof; and a non-aqueous discontinuous phase. The composition is made by mixing water and a fruit or vegetable juice to make an aqueous juice phase; preparing a nonaqueous mixture of at least one thickener; at least one hydrophobic solvent, wax or oil; and an emulsifier to make a hydrophobic phase; mixing the hydrophobic phase and the aqueous juice phase to form a uniform emulsion; and optionally adding at least one component selected from the group consisting of tanning accelerators, bronzers, and a mixture thereof to the uniform emulsion.

### BACKGROUND OF THE INVENTION

1. Field of the Invention

This invention relates generally to a cosmetic tanning formulation.

2. Background of the Invention

A natural tan can require prolonged exposure to ultraviolet (UV) radiation, particularly when using indoor tanning beds.

Indoor tanning equipment typically employs ultraviolet lights positioned beneath and above a planar surface which is transparent to ultraviolet radiation have become increasingly popular for acquiring a tan. Generally, such tanning equipment employs a planar surface made out of a material transparent to ultraviolet radiation, such as acrylic Plexiglas. The source of ultraviolet radiation is positioned above and below the UVT Plexiglas on which the person lies.

Lotions and compositions disclosed in the prior art fail to disclose the aspects of the current invention. In addition, indoor tanning products that claim to be an aqueous juice based composition are not known in the prior art.

What is needed is a composition for use in indoor tanning equipment that: (1) can be spread topically on the skin, (2) will not interfere with the passage of ultraviolet radiation of the wavelength needed for effective tanning, (3) will not contaminate the surface of the equipment, and (4) enhances UV exposure.Tanning accelerators in the form of lotions or pills that contain the amino acid tyrosine are alleged to stimulate and increase melanin formation, thereby accelerating the tanning process. These tanning accelerators are used in conjunction with UV exposure.

Tanning accelerators in the form of lotions or pills that contain the amino acid tyrosine are alleged to stimulate and increase melanin formation, thereby accelerating the tanning process. These tanning accelerators are used in conjunction with UV exposure.

Enzymes within fruits and vegetables are known to convert tyrosine into melanin. It is an object of this invention to improve the effectiveness of tanning accelerators by incorporating an active enzyme-containing juice component which increases melanin formation.

Additionally, many cosmetic or topical formulations contain colorant ingredients or active ingredients which interfere with the passage of U.V. radiation. These colorants or active ingredients may prevent U.V. radiation from reaching the melanocytes in the skin by reflecting U.V. radiation or by absorbing U.V. radiation. This prevents the topical formulation from effectively allowing or promoting tanning. This problem would be solved by a preparation which is substantially transparent to U.V. radiation.

### SUMMARY OF THE INVENTION

In light of the present need for improved tanning compositions which offer a prolonged warming sensation while remaining substantially transparent to U.V. radiation, a brief summary of various exemplary embodiments is presented. Some simplifications and omission may be made in the following summary, which is intended to highlight and introduce some aspects of the various exemplary embodiments, but not to limit its scope. Detailed descriptions of a preferred exemplary embodiment adequate to allow those of ordinary skill in the art to make and use the invention concepts will follow in later sections.

One embodiment of the invention relates to a topical tanning composition comprises an aqueous continuous phase comprising a fruit or vegetable juice selected from the group consisting of an orange juice, grapefruit juice, apple juice, grape juice, pomegranate juice, carrot juice or a mixture thereof. The topical/cosmetic application of this composition to the skin prior to exposing the skin to UV rays of indoor tanning equipment enhances UV exposure by the formula's unique aqueous juice composition. The composition is also cosmetically pleasant to the consumer and minimizes smears and oil residue on the indoor tanning equipment.

The composition may also comprise a tanning accelerator, a bronzer, a source of tyrosine, mixtures thereof and derivatives thereof.

The composition does not contaminate the surface of the equipment or inhibit the passage of ultraviolet radiation of the wavelength needed for effective tanning. The nature of the composition leads to consistency for each application with uniform distribution to the skin without interference of UV rays. The composition provides particular esthetics of long lasting skin feel with quick rub-in and silky after-feel.

One embodiment of the invention relates to a topical tanning composition, comprising an aqueous continuous phase comprising water; a fruit or vegetable juice; and a non-aqueous discontinuous phase. The composition may further comprise at least one optional compound selected from the group consisting of tanning accelerators, bronzers, and a mixture thereof

In an additional embodiment of the invention, a tanning composition is made by mixing water and a fruit or vegetable juice to make an aqueous juice phase; preparing a nonaqueous mixture of at least one polymeric thickener; at least one hydrophobic solvent or oil, and an anionic, cationic, or nonionic emulsifier to make a hydrophobic phase; and mixing the hydrophobic phase and the aqueous juice phase to form a uniform emulsion. Subsequently, at least one compound selected from the group consisting of tanning accelerators, bronzers, and a mixture thereof may optionally be added to the uniform emulsion.

### BRIEF DESCRIPTION OF THE PREFFERED EMBODIMENTS

Although the various exemplary embodiments have been described in detail with particular reference to certain exemplary aspects thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

In light of the current need for cosmetic tanning formulations which give rise to a deeper, richer color, various exemplary embodiments of the invention disclosed herein are directed to a topical tanning composition for use with Indoor Tanning Equipment containing an aqueous continuous phase and a non-aqueous discontinuous phase. The aqueous phase contains a fruit or vegetable juice component, which may comprise orange juice, grapefruit juice, apple juice, grape juice, pomegranate juice, carrot juice or a mixture thereof. The juices used are of value because they contain oxidizing enzymes such as tyrosinase or polyphenol oxidase (PPO). These enzymes are important in the production of melanin from the phenolic amino acids tyrosine and 3,4-dihydroxyphenylalanine (DOPA), as depicted in Scheme 1. The phenolic amino acids undergo oxidation in the presence of air and tyrosinase or polyphenol oxidase to produce ortho quinones, which then undergo cyclization and polymerization to produce melanin. The tanning formulation, when applied, is substantially transparent to ultraviolet radiation.

In light of the current need for cosmetic tanning formulations which give rise to a deeper, richer color, various exemplary embodiments of the invention disclosed herein are directed to a tanning cream or emulsion containing an aqueous continuous phase and a non-aqueous discontinuous phase. The aqueous phase contains a fruit or vegetable juice component. The juice component may comprise orange juice, grapefruit juice, apple juice, grape juice, pomegranate juice, carrot juice, banana juice, beet juice, blueberry juice, cherry juice, cranberry juice, lime juice, key lime juice, lemon juice, kiwi juice, mango juice, passion fruit juice, peach juice, pineapple juice, pomegranate juice, pumpkin juice, raspberry juice, strawberry juice, tomato juice, or a mixture thereof. In some exemplary embodiments, all or part of the juice component may be replaced with honey, sugar cane juice, or molasses. In certain exemplary embodiments, the juices may contain oxidizing enzymes such as tyrosinase or polyphenol oxidase (PPO). These enzymes are believed to be useful in the production of melanin from the phenolic amino acids tyrosine and 3,4-dihydroxyphenylalanine (DOPA), as depicted in Scheme 1. The phenolic amino acids undergo oxidation in the presence of air and tyrosinase or polyphenol oxidase to produce ortho quinones, which then undergo cyclization and polymerization to produce melanin. The tanning formulation, when applied, is substantially transparent to ultraviolet radiation.

In certain exemplary embodiments, the juices contain phenolic pigments. These phenolic pigments, like tyrosine and DOPA, are believed to undergo oxidation in the presence of air and tyrosinase or polyphenol oxidase. This process, often referred to as enzymatic browning, results in formation of dark pigments, often referred to as vegetable melanin. These pigments, when the juice is included in a cosmetic tanning formulation, help impart a deep, rich brown color to the skin.

Additionally, the aqueous juice component of the compositions described herein serve to increase the water content of the skin and to retard moisture loss from the skin over time.

The juices are prepared by squeezing or pureeing raw fruits or vegetables. In certain exemplary embodiments, fruits and vegetables may be juiced by cold-pressing them throu a microfine screen to remove seeds and stems. In certain exemplary embodiments, juices prepared from organically grown fruits or vegetables, produced without pesticides, may be used. Although they may be used in a raw, unprocessed form, it is preferred that they be subjected to further processing. This processing typically includes a step of filtering the juices to remove pulp and other solids to produce a clear, filtered juice. If unprocessed juices or filtered juices are used without further processing, the aqueous continuous phase will typically contain from 5% by weight to 15% by weight of juice with the balance being water. The filtered juices may also undergo further processing. The most common form of processing is preparation of a concentrate. Although concentrates can be prepared by evaporation of water by exposing the juice to reduced pressure and/or elevated temperature, this is very energy- and cost-intensive. Additionally, elevated temperature can denature enzymes such as tyrosinase or polyphenol oxidase, potentially reducing the effectiveness of the juice.

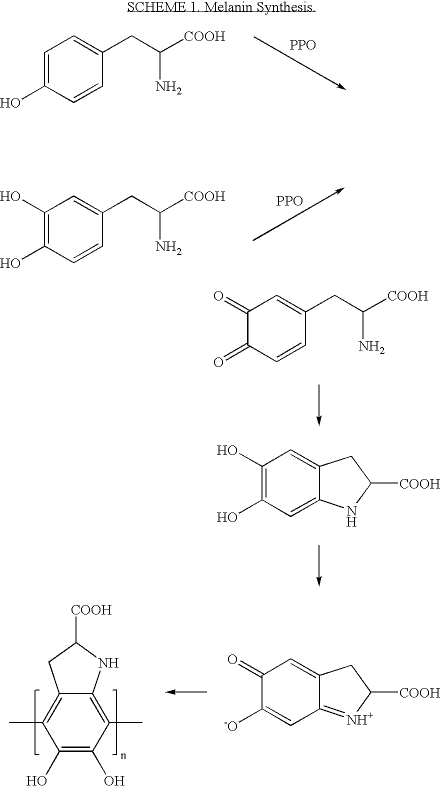

In various exemplary embodiments, a juice concentrate may be prepared in powdered form and dissolved in the aqueous phase. In various other exemplary embodiments, the concentrate may be prepared by a process of reverse osmosis, without exposing the juice to high temperatures or organic solvents which could denature the enzymes in the juice. Filtered juice is pumped under pressure past a semipermeable membrane which allows water molecules to pass therethrough. The membrane retains the enzymes, pigments, and other solutes in the juice. On the opposite side of the membrane, water flows past the membrane at a reduced pressure, relative to the juice. Water passes from the pressurized juice side of the membrane into the less pressurized water, concentrating the juice. The juice is typically concentrated to between 5% and 50% of its original volume, preferably between 10% and 40% of its original volume, more preferably between 15% and 35% of its original volume.

In various exemplary embodiments, the semipermeable membrane allows only water molecules to pass therethrough. In various exemplary embodiments, the reverse osmosis process may be performed with a semipermeable membrane which allows water molecules and mono- and disaccharides to pass therethrough, while retaining the enzymes, pigments, and other solutes in the juice. This allows preparation of a reduced sugar juice. This has the advantage of reduced likelihood of a sensation of tackiness or stickiness on the skin after application of a cream or lotion containing the juice to the skin.

In various exemplary embodiments, commercially available juices may be used as the juice component. These juices may be bottled juices, liquid juice concentrates, powdered juice concentrates, or frozen juice concentrates. Commercially available juice products include organic 100% Concord grape juice, organic 100% white grape juice, organic 100% apple juice, organic 100% orange juice, organic 100% grapefruit juice, organic 100% carrot juice, mixtures thereof, and concentrates thereof.

Once the juice has been prepared, an aqueous continuous phase is prepared by mixing from 0.5% to 5% by weight of a juice concentrate with from 99.5% to 95% by weight of water. Alternatively, the aqueous continuous phase may be prepared by mixing from 5% to 15% by weight of a filtered unconcentrated juice with from 95% to 85% by weight of water. The aqueous continuous phase is then mixed with a nonaqueous discontinuous phase to produce an emulsion. Up to 20% by weight of polar cosolvents may be present. Examples of cosolvents include lower alcohols, glycerine, ethylene glycol, propylene glycol, butylene glycol, mono- or diethers of glycerin, aliphatic glycol ethers, and aromatic glycol ethers. The cosolvents may be added to the aqueous phase prior to formation of the emulsion, or to the emulsion after mixing the aqueous continuous phase and the nonaqueous discontinuous phase.

The compositions described herein are generally formed as emulsions. Emulsions are generally formed from at least two liquid phases which are immiscible so that at least one of the phases is dispersed in fine form throughout the other phase(s). While emulsions are often formed by many components in complex relationships, they can be broadly classified as either oil-in-water or water-in-oil emulsions, depending on which of the phases comprises the dispersed inner phase and which is the continuous outer phase. In this context, an oil may be defined as any material immiscible with water and capable of forming an emulsion with water. Various agents are used to retard or inhibit the separation of emulsions into their constituent phases and these agents may determine the type of emulsion formed. Additionally, the nature of the emulsion can be reversed depending on the agent employed. The preferred embodiment of the composition is a stabilized oil-in-water (“o/w”) emulsion.

The nonaqueous phase contains at least one hydrophobic or water-repellant solvent, wax or oil. Suitable oils include plant-derived triglycerides, such as peanut, soya bean, sunflower, sesame, coconut, olive and other vegetable oils; and silicone oils. Suitable waxes include C12-C26 fatty alcohols; C12-C26 fatty acids; alkyl esters of C12-C26 fatty acids; C2-C5 linear or branched glycol diesters of C12-C26 fatty acids; C12-C26 fatty alcohol esters of carboxylic acids; beeswax; camauba wax; and paraffin wax. When the nonaqueous and aqueous phases are combined, at least one emulsifier or surfactant is present. The emulsifier or surfactant may be anionic, cationic, nonionic, or zwitterionic. Mixtures of nonionic surfactants with either cationic or nonionic surfactants may also be used.

The anionic surfactants employed may be salts of fatty acids (for example alkaline salts or organic salts such as amine salts), the said fatty acids having, for example, from 12 to 18 carbon atoms and being able to have a double bond as in the case of oleic acid; alkaline salts or organic base salts of alkylsulfates and alkylsulfonates having 12 to 18 carbon atoms; alkylsulfosuccinic acids whose alkyl chain contains from 6 to 18 carbon atoms; anionic sphingolipids; and alkylphenylsulfonic acids whose alkyl chain contains from 6 to 18 carbon atoms. They may also be alkyl ether sulfates, in particular the sulfonation products of polyalkoxylated fatty alcohols and polyalkoxylated alkyl phenols in which the aliphatic chain contains from 6 to 20 carbon atoms and the polyalkoxylated chain from 1 to 30 oxyalkylene units, in particular oxyethylene, oxypropylene or oxybutylene. Also, C6-C26 monoalkyltartrates may be used as anionic surfactants.

The nonionic surfactants are principally polyalkoxylated and/or polyglycerolated surfactants. They are principally polyalkoxylated and/or polyglycerolated fatty acids or amides of fatty acids; polyalkoxylated and/or polyglycerolated fatty alcohols or alkylphenols; polyalkoxylated and/or polyglycerolated esters of fatty acids and polyols; polyalkoxylated and/or polyglycerolated 1,2- or 1,3-alkanediols or alkenediols; polyalkoxylated and/or polyglycerolated alkylethers of 1,2- or 1,3-alkanediols or alkenediols; C6-C26 dialkyl tartrates; and nonionic sphingolipids. For example, the fatty acids or alcohols, optionally unsaturated, have 12-24 carbon atoms, the alkyl chain of the alkylphenols has 6 to 16 carbon atoms, the alkanediols or alkenediols have from 9 to 24 carbon atoms, the alkyl of the alkylethers has from 4 to 20 carbon atoms, and the number of oxyalkylene units or of (CH₂CHOHCH₂O) units can range from 2 to 40. The polyalkoxylated nonionic derivatives are principally polyoxyethylenated, optionally polyoxypropylenated derivatives. Ethoxylated fatty acid esters of sorbitan, such as Polysorbate 20, may also be used as nonionic surfactants.

The cationic surfactants can be chosen from quaternary ammonium derivatives such as C6-C18 dialkyldimethylammonium salts; C6-C 18 alkyltrimethylammonium salts; and C6-C18 alkyldimethylbenzylammonium salts. Quatemary phosphonium salts may also be used

Polymeric thickeners such as polysaccharide starches or vegetable gums; proteins such as albumin, gelatin, hydrolyzed vegetable protein, or collagen; and polyacrylate or polyacrylamide thickeners may be added to the emulsion to increase the viscosity. Other thickeners can be polyols like sorbitol, xylitol, dextran and maltitol, or polymeric polyols like polydextrose or natural extracts like quillaia.

The aqueous phase containing the juice is combined with the oil or wax, the surfactant, and the emulsifier, and mixed to produce a smooth and uniform cream or gel. Additional active agents are then added to the cream or gel. Various dermatologically acceptable fragrances and colorants may be added to the cream or gel. Antibacterial and antifungal agents such as triclosan may also be added.

Additional active components which are effective as tanning accelerators or melanin accelerators may be added to the lotion. In various exemplary embodiments, these tanning accelerators include tyrosine and/or derivatives thereof. Such derivatives include N-acetyl tyrosine, N-acetyl o-dihydroxymethylsilyltyrosine (Tyrosilane C®), N-caproyl tyrosine (Tyrostan®), glucose tyrosinate, 3,4-dihydroxyphenylalanine, and alkali metal salts thereof. The tyrosine-derived pigment melanin may also be added. Sulfate salts selected from the group consisting of magnesium sulfate, manganese sulfate, copper sulfate, zinc sulfate, and a combination thereof may also be used as tanning accelerators, alone or in combination with hydrolyzed algin. Coelus Forskohlii Extract and Micrococcus Lysate are effective as melanin accelerators. A wide variety of vegetable extracts may be used as melanin accelarators. Such extracts include citrus aurantium dulcis (orange) fruit extract, hydrolyzed citrus aurantium dulcis fruit extract, citrus aurantium dulcis peel extract, cocoa fruit extract, and vanilla planifolia fruit extract. Vegetable melanin is also effective as a melanin accelerator. Extracts of Saccharomyces yeasts fermented in the presence of magnesium, copper and/or zinc ions are effective as melanin accelerators.

Other tanning accelerators may also be added. For example, tanning accelerator peptides may be added. The tanning accelerator peptides may be selected from the group consisting of α-melanocyte stimulating hormone, β-melanocyte stimulating hormone, γ-melanocyte stimulating hormone and analogs thereof. The melanocyte-stimulating hormones (collectively referred to as MSH) are a class of peptide hormones. They stimulate the production and release of melanin (melanogenesis) by melanocytes in skin and hair. MSH is also produced by a subpopulation of neurons in the arcuate nucleus of the hypothalamus. MSH causes a darkening of skin pignentation. Synthetic peptides which are structurally related to MSH, or which perform the same function as MSH, may be used to partially or completely replace MSH. An example of such a peptide is acetyl hexapeptide-1, commercially available as Melitane.

The cream or lotion may contain a wide variety of extracts of plants and microorganisms which act as bronzers. These bronzers are plant extracts which give a suntan-like color to skin. For example, black walnut leaf extracts and/or kukui nut husk extracts may be added to the formulation as bronzers; in addition to their cosmetic effect, these bronzers have additional health benefits. Walnut leaves contain astringent tannins which cross-link skin cells, making them impermeable to allergens and infectious microorganisms. Walnut leaves also contain antibacterial agents. Kukui oil assists the skin in maintaining its water barrier function, and is useful in the topical treatment of viral infections. Fruit, flower, and/or leaf extracts of henna and Gymnema sylvestre leaf extracts are other preferred bronzers. Other extracts useful as bronzers include Musa Sapientum (Banana) Fruit Extract, carrot seed oil, Cucumis Melo (Melon) Fruit Extract, mustard, black tea extract, chicory root extract, hibiscus extracts, wasabi extracts, red beet extracts, paprika extracts, red clover extracts, and vegetable melanin. Other bronzing agents which may be used include melanin, erythrulose, dihydroxyacetone, a mixture of erythrulose and dihydroxyacetone, and caramel color.

A variety of antioxidant extracts may also be added to the cream or lotion. These antioxidant extracts include Ligustrum Lucidum Fruit Extract, Hypericum Perforatum Flower, Leaf and Stem Extracts, Phellodendron Amurense Bark Extract, Orobanche Rapum Extract, Helianthus Annuus (Sunflower) Seed Extract, Leontopodium Alpinum Flower and Leaf Extracts, and Bixa Orellana Seed Extract.

Nucleotides such as adenosine monophosphate, adenosine triphosphate, and cyclic adenosine monophosphate, may also be added to the tanning formulation. Adenosine is a potent topical anti-inflammatory agent, acting at its four G-protein coupled receptors. Adenosine therefore may be useful in treating inflammation from excessive sun exposure.

The formulation may contain transdermally available vitamins, including vitamin A, vitamin C, and vitamin E. An analog of Vitamin A, Retinyl Palmitate Polypeptide, helps to reverse photo-damage to the skin from the sun. A useful form of vitamin C is L-ascorbic acid polypeptide complex, a water stable, soluble form of Vitamin C, which is able to penetrate the outer layers of the skin. On topical application, it enters the melanocyte cells in the skin. Vitamin E is used in the form of α-tocopheryl nicotinate. Soy proteins and/or amino acids may also be added to enhance protein formation in the skin. Vitamins and amino acids also enhance tanning by moisturizing the skin and contributing to a uniform tan color.

Other additives can include, depending on the use, glycols, sunscreen agents, humectants, preservatives, such as known parabens, emollients, occlusive agents, and esters. Depending on the use of the composition, the other additives may be dispersible in the oil or water phase. Preferably, the additives are incorporated in the appropriate phase before the oil mix is charged into the water phase. In such instances, the oil or water phase can be separately mixed or blended with the additives to produce a uniform phase prior to the mixing of the oil and water phase.

The aqueous phase containing the juice is combined with the oil or wax, the surfactant, and the emulsifier, and mixed to produce a smooth and uniform cream or gel. Additional active agents are then added to the cream or gel. Various dermatologically acceptable acceptable fragrances and colorants may be added to the cream or gel. Deodorant or odor neutralizing agents such as Triclosan or Lemongrass derivative Citronellyl Methylcrotonate may also be added.

The preferred emulsion can be produced by the process comprising the following steps forming an aqueous phase under low shear conditions. In producing the stabilized emulsion of the preferred embodiment, the oil and water phases are formed either as a single phase process or separately. For example, in a first vessel, the water and water compatible agents are mixed together under low shear conditions at room temperature. With a cold process to preserve the integrity of the aqueous juice phase the oil phase is introduced, preferably at a sufficient shear so that satisfactory dispersion of the oil phase can be achieved and the emulsion formed.

GENERAL PROCEDURE

| | |
|---|---|
| Ingredient | W/W |

Phase 1

| | | |
|---|---|---|
| Fruit or Vegetable Juice from Concentrate | 0.2-5 | parts |
| Polysorbate 20 | 0.1-5 | parts |
| Water | 65-100 | parts |

Phase 2

| | | |
|---|---|---|
| 1.4 parts polyacrylamide; | 1-10 | parts |
| 0.875 parts of a C13-C14 isoparaffin; | | |
| <1 part PEG-7 lauryl ether nonionic surfactant | | |
| Dimethicone Crosspolymer/Dimethicone (Dow | 1-15 | parts |
| Corning 9041) | | |

Phase 3

| | | |
|---|---|---|
| Odor Neutralizing Agents | 0.1-2 | |
| Hydrogenated Polydecene | 1-10 | parts |
| Neopentyl Glycol Dicaprylate | 1-10 | parts |
| Botanical Extracts | 0-10 | parts |

Phase 4

| | | |
|---|---|---|
| Preservative & Fragrance | 0-10 | parts |
| Color & Additives | 0-5 | parts |

Phase 1 was individually formed at room temperature by combining the juice, the Polysorbate 20, and the water and stirring until homogeneous. Phase 2 was prepared by mixing the polyacrylamide/isoparaffin/surfactant phase with the dimethicone mixture and mixing until smooth. Phase 2 was added to Phase 1 at ambient temperature and the mixture of the phases was being mixed with high shear mixing for 1 hour to form, an emulsion. Phase 3 was prepared by mixing odor neutralizing agents with hydrogenated polydecene wax and Neopentyl Glycol Dicaprylate surfactant. Desired antioxidant botanical extracts, bronzing botanical extracts, tanning accelerator extracts, and tanning accelerator peptides are combined with the hydrogenated polydecene mixture, and the polydecene mixture is added to the emulsion at ambient temperature while mixing with high shear mixing. Additional extracts may be combined with the silicone composition (Phase 2) or with the aqueous juice mixture (Phase 1), either before or after mixing Phases 1 and 2. Phase 4, containing preservatives, fragrances, colorants, and other optional additives, was added to the emulsion with mixing at high shear. The final emulsion was mixed with lower shear until a smooth consistency was obtained.

The resulting cream was easy to apply, had good tactile properties but no oily feel and exhibited excellent stability.

### EXAMPLE 1

In a first step of preparing a topical tanning cream, a mixture of 1.4 parts of a polyacrylamide thickener, 0.875 parts of a C13-C14 isoparaffin, and less than 1 part of a PEG-7 lauryl ether nonionic surfactant are added to 7 parts of a mixture of dimethicone and a vinyl-substituted crosslinked dimethicone. The resulting silicone mixture is blended until smooth. Next, 1 part by weight of orange juice concentrate is mixed with 99 parts by weight of water to form an aqueous juice base. 75.75 parts of the juice base are then mixed with 0.5 parts of Polysorbate 20, and the resulting mixture is combined with the silicone mixture and mixed until a smooth aqueous juice emulsion is formed.

Next, 0.2 parts of triclosan, 0.4000 parts citronellyl methylcrotonate, 3.0000 parts hydrogenated polydecene; and 7.0000 parts neopentyl glycol dicaprylate are individually added to the juice-containing emulsion with stirring until the mixture isrethickened and uniform. 1 part of a preservative, 1,65 parts of a fragrance are then mixed into the emulsion with stirring, and the resulting juice containing emulsion is stirred until uniform. The final emulsion is a thickened balm or lotion. If desired, the lotion may be colored with a small amount of a 1% solution of the colorant yellow 5, red 40, and/or green 5.

### EXAMPLE 2

In a first step of preparing a topical tanning cream, 3 parts of Sepigel 305, a thickener composition containing a mixture of 1.4 parts of a polyacrylamide thickener, 0.875 parts of a C13-C14 isoparaffin, and less than 1 part of a PEG-7 lauryl ether nonionic surfactant, are added to 7 parts of a mixture of dimethicone and a vinyl-substituted crosslinked dimethicone. The resulting silicone mixture is blended until smooth. Next, 1 part by weight of orange juice concentrate is mixed with 99 parts by weight of water to form an aqueous juice base. 71.3 parts of the juice base are then mixed with 0.5 parts of Polysorbate 20, and the resulting mixture is combined with 10 parts of the silicone mixture and mixed until a smooth aqueous juice emulsion is formed.

In a second step, 0.01 to 4 parts [IS THIS CORRECT?] of a premix composition containing tyrosine is prepared by mixing melanin, acetyl tyrosine, methylsilanol acetyltyrosine, adenosine triphosphate, hydrolyzed vegetable protein, hydrolyzed citrus aurantium dulcis fruit extract, musa sapientum (banana) fruit extract, butylene glycol, and water. To the resulting composition is added 0.3 parts of a composition containing butylene glycol, acetyl tyrosine, hydrolyzed vegetable protein, and adenosine triphosphate. Next, 0.3 parts methylsilanol tyrosine; 0.2 parts melanin; and 0.1 parts of an aqueous glycerin solution of Musa Sapientum (banana) fruit extract are added to the tyrosine containing composition.

Next, a mixture of water, soy amino acids, saccharomyces lysate extract, nucleotides, ascorbic acid polypeptide, retinyl palmitate polypeptide, tocopherol nicotinate, cyclic amp, coelus forskohlii extract, and micrococcus lysate is prepared, and 0.1 parts of this composition are added to the tyrosine containing composition. Finally, 0.1 parts potassium N-caproyl tyrosine, 0.25 parts black walnut extract, 0.25 parts kukui nut extract, and 0.2 parts triclosan are added to the tyrosine containing premix composition. The resulting tyrosine containing premix composition is thoroughly mixed and slowly added to the smooth aqueous juice emulsion. The resulting mixture is stirred until thickened and uniform.

After this, 0.7000 parts by weight of a glycerin solution of Ligustrum Lucidum fruit extract; 0.3000 parts by weight of an aqueous glycerin solution of Hypericum Perforatum flower, leaf, and/or stem extracts; and 0.4000 parts by weight of an aqueous glycerin solution of Phellodendron Amurense bark extract are mixed and added to the emulsion with stirring.

In the next step, a mixture is formed which contains 0.0001 parts by weight of an aqueous butylene glycol solution of Orobanche Rapum extract; 0.0001 parts by weight of Saccharomyces Ferment Lysate Filtrate; 0.0001 parts by weight of a cyclomethicone-based composition containing sphingolipids, di-C14-15 alkyl tartrate, Helianthus Annuus (Sunflower) seed extract, and Leontopodium Alpinum flowerand/or leaf extracts; 0.0001 parts by weight of an aqueous glycerin solution of Cucumis Melo (Melon) fruit extract; and 0.0001 parts by weight of an aqueous glycerin solution of Bixa Orellana seed extract. This mixture of extracts is added to the juice-containing emulsion with stirring.

A wax mixture of 0.4000 parts citronellyl methylcrotonate, 3.0000 parts hydrogenated polydecene; and 7.0000 parts neopentyl glycol dicaprylate is next added to the juice-containing emulsion with stirring. An organic solvent mixture of 0.6 parts phenoxyethanol, 0.3 parts pentylene glycol, and 1 part of a mixure of phenoxyethanol and ethylhexylglycerin is mixed into the emulsion next. Finally, a solution of 0.05 parts D-chiro-inositol in 0.5 parts water is added to the emulsion, and the resulting juice containing emulsion is stirred until uniform.

What is claimed is:

1) A topical tanning composition, comprising:
a) an aqueous continuous phase comprising water; and at least one fruit or vegetable juice;
b) at least one optional component selected from the group consisting of tanning accelerators, bronzers, and a mixture thereof; and
c) a non-aqueous discontinuous phase.

2) A tanning composition according to claim 1, wherein the fruit or vegetable juice is selected from the group consisting of orange juice, grapefruit juice, apple juice, grape juice, pomegranate juice, carrot juice, banana juice, beet juice, blueberry juice, cherry juice, cranberry juice, lime juice, key lime juice, lemon juice, kiwi juice, mango juice, passion fruit juice, peach juice, pineapple juice, pomegranate juice, pumpkin juice, raspberry juice, strawberry juice, tomato juice, honey, sugar cane juice, molasses, or a mixture thereof.

3) A tanning composition according to claim 1, wherein the fruit or vegetable juice is selected from the group consisting of orange juice, grapefruit juice, apple juice, grape juice, pomegranate juice, carrot juice, or a mixture thereof.

4) A tanning composition according to claim 1, wherein the aqueous continuous phase comprises 95.0-99.5% by weight of water; and 0.5-5.0% by weight of a concentrated form of the fruit or vegetable juice.

5) A topical tanning composition according to claim 2, wherein the topical tanning composition is transparent to ultraviolet light.

6) A topical tanning composition according to claim 4, wherein the concentrated form of the fruit or vegetable juice is a liquid concentrate, a powdered concentrate.

7) A topical tanning composition according to claim 1, wherein the fruit or vegetable juice is prepared from organically grown fruits or vegetables.

8) A topical tanning composition according to claim 1, wherein the at least one optional component is present, and comprises a bronzer selected from the group consisting of black walnut extract, henna extract, kukui nut husk extract, and gymnema extract.

9) A topical tanning composition according to claim 1, wherein the at least one optional component is present, and comprises:
i) tyrosine, a tyrosine derivative, or a mixture thereof;
ii) α-melanocyte stimulating hormone, β-melanocyte stimulating hormone, γ-melanocyte stimulating hormone, and analogs thereof; or iii) a combination of (i) and (ii).

10) A topical tanning composition according to claim 1, wherein the nonaqueous phase comprises a wax or oil selected from the group consisting of peanut oil, soya bean oil, sunflower oil, sesame oil, coconut oil, olive oil, silicone oils, C12-C26 fatty alcohols, C12-C26 fatty acids, alkyl esters of C12-C26 fatty acids, C2-C5 linear or branched glycol diesters of C12-C26 fatty acids, C12-C26 fatty alcohol esters of carboxylic acids, beeswax; carnauba wax; and paraffin wax.

11) A topical tanning composition according to claim 1, wherein the composition further comprises an anionic surfactant, a cationic surfactant, a nonionic surfactant, a zwitterionic surfactant, or a mixture of a nonionic surfactant with a cationic surfactant or a nonionic surfactant.

12) A topical tanning composition according to claim 1, wherein the composition further comprises at least one thickener selected from the group consisting of polysaccharide starches; vegetable gums; albumin; gelatin; hydrolyzed vegetable protein; collagen; polyacrylate thickeners; polyacrylamide thickeners; sorbitol; xylitol; dextran; maltitol; polydextrose; and quillaia.

13) A method of making a tanning composition, comprising:
a) mixing water and a fruit or vegetable juice to make an aqueous juice phase;
b) preparing a nonaqueous mixture of at least one thickener; at least one hydrophobic solvent, wax or oil; and an emulsifier to make a hydrophobic phase;
c) mixing the hydrophobic phase and the aqueous juice phase to form a uniform emulsion; and
d) optionally adding at least one component selected from the group consisting of tanning accelerators, bronzers, and a mixture thereof to the uniform emulsion.

14) The method of claim 13, wherein the at least one component comprises a bronzer selected from the group consisting of black walnut extract, henna extract, kukui nut husk extract, and gymnema extract.

15) The method of claim 13, wherein the at least one component comprises:
i) tyrosine, a tyrosine derivative, or a mixture thereof;
ii) α-melanocyte stimulating hormone, β-melanocyte stimulating hormone, γ-melanocyte stimulating hormone, and analogs thereof; or
iii) a combination of (i) and (ii).

16) The method of claim 13, wherein the at least one component comprises a mixture of i) tyrosine or a derivative thereof; and ii) black walnut extract, henna extract, kukui nut husk extract, gymnema extract or a mixture thereof.

17) The method of claim 13, wherein the fruit or vegetable juice is selected from the group consisting of orange juice, grapefruit juice, apple juice, grape juice, pomegranate juice, carrot juice, banana juice, beet juice, blueberry juice, cherry juice, cranberry juice, lime juice, key lime juice, lemon juice, kiwi juice, mango juice, passion fruit juice, peach juice, pineapple juice, pomegranate juice, pumpkin juice, raspberry juice, strawberry juice, tomato juice, honey, sugar cane juice, molasses, or a mixture thereof.

18) The method of claim 13, wherein the fruit or vegetable juice is a fruit or vegetable juice concentrate.

19) The method of claim 13, wherein the aqueous juice phase comprises 95.0-99.5% by weight of water; and 0.5-5.0% by weight of a concentrated form of the fruit or vegetable juice.

20) A topical tanning composition according to claim 13, wherein the at least one hydrophobic solvent, wax or oil is selected from the group consisting of peanut oil, soya bean oil, sunflower oil, sesame oil, coconut oil, olive oil, silicone oils, C12-C26 fatty alcohols, C12-C26 fatty acids, alkyl esters of C12-C26 fatty acids, C2-C5 linear or branched glycol diesters of C12-C26 fatty acids, C12-C26 fatty alcohol esters of carboxylic acids, beeswax; carnauba wax; and paraffin wax.

21) A topical tanning composition according to claim 13, wherein the emulsifier comprises an anionic surfactant, a cationic surfactant, a nonionic surfactant, a zwitterionic surfactant, or a mixture of a nonionic surfactant with a cationic surfactant or a nonionic surfactant.

22) A topical tanning composition according to claim 13, wherein the thickener is selected from the group consisting of polysaccharide starches; vegetable gums; albumin; gelatin; hydrolyzed vegetable protein; collagen; polyacrylate thickeners; polyacrylamide thickeners; sorbitol; xylitol; dextran; maltitol; polydextrose; and quillaia.
